SEQUENCE LISTING (40 sequences)

SEQ ID NO: 1 - protein, 267 aa, Artificial sequence
  synthesized
MAPRRARGCRTLGLPALLLLLLLRPPATRGITCPPPMSVEHADIWVKSYSLYSRERYICNSGFKRKAGTSSLTECVLNKATNVAHWTTPSLKCIRDPALVHQRPAPPSTVTTAGVTPQPESLSPSGKEPAASSPSSNNTAATTAAIVPGSQLMPSKSPSTGTTEISSHESSHGTPSQTTAKNWELTASASHQPPGVYPQGHSDTTVAISTSTVLLCGLSAVSLLACYLKSRQTPPLASVEMEAMEALPVTWGTSSRDEDLENCSHHL

SEQ ID NO: 2 - DNA, 801 bp, Artificial sequence
  IL15 RNA protein amino acid sequence
atggctcctagaagagctagaggatgtagaactttgggcctccctgctctcttgctgcttctgctgttgaggccgcccgctactagaggaataacctgtccacccccaatgtccgtcgaacacgccgatatttgggtgaagagttattcactgtatagccgggaacgctacatttgtaacagcggattcaaacgaaaagccggcacttcctctttgactgagtgcgttctgaacaaagccacgaatgtcgcacactggaccacgccttcattgaagtgtattcgcgatccagcactggttcaccaaaggccggctcctccatctactgttacaacagctggcgtgacaccacagcctgagtccctgtctccttctgggaaggaacccgctgcctcaagcccttcctctaacaacaccgcagctacaacagccgcgatcgtgccaggctcacagctgatgcctagcaaaagcccatctactggcaccactgagatctcaagccacgaatcctcacacggaaccccttcacagactactgccaagaattgggagctgactgcttcagcaagccaccaacctcctggagtctaccctcagggccacagtgacactaccgtggccatttctacatccaccgtgctgctgtgtggtctttccgccgtctctctcttggcatgttacctgaaatcaagacaaactccaccactggccagcgtggaaatggaagctatggaggcactcccagtgacctgggggacctcttcaagagatgaagacttggagaattgctcccaccacctg

SEQ ID NO: 3 - protein, 135 aa, Artificial sequence [sequence omitted]

SEQ ID NO: 4 - DNA, 405 bp, Artificial sequence
  IL-15 DNA sequence
atggtccttggaacaatcgacctctgttcatgctttagcgccggcctgccaaaaactgaagctaactgggtaaatgtgatttccgacctcaaaaagatcgaagacctgattcagtctatgcatattgacgctacactgtatactgaaagcgacgttcacccaagctgtaaggtgacagctatgaagtgtttccttctggaactccaggtgattagtcttgagagcggcgacgcctccatccacgacacagtagagaatctgattattctggctaataattctctgtctagcaacggaaacgtgactgagtcagggtgtaaagagtgtgaggagctggagaaaaagaatataaaggaattcttgcagtccttcgtccacattgtgcaaatgtttattaataccagc

SEQ ID NO: 5 - protein, 20 aa, Artificial sequence
  Amino acid sequence of linker connecting IL-15 and IL-15 RNA
SGGSGGGGSGGGSGGGGSGG

SEQ ID NO: 6 - DNA, 60 bp, Artificial sequence
  DNA sequence of linker connecting IL-15 and IL-15 RNA
tctggcggatcaggtggaggtggttctggtggtggttcaggaggaggaggtagtggaggt

SEQ ID NO: 7 - protein, 422 aa, Artificial sequence
  IL-15-IL-15 RNA fusion protein amino acid sequence
MVLGTIDLCSCFSAGLPKTEANWVNVISDLKKIEDLIQSMHIDATLYTESDVHPSCKVTAMKCFLLELQVISLESGDASIHDTVENLIILANNSLSSNGNVTESGCKECEELEKKNIKEFLQSFVHIVQMFINTSSGGSGGGGSGGGSGGGGSGGMAPRRARGCRTLGLPALLLLLLLRPPATRGITCPPPMSVEHADIWVKSYSLYSRERYICNSGFKRKAGTSSLTECVLNKATNVAHWTTPSLKCIRDPALVHQRPAPPSTVTTAGVTPQPESLSPSGKEPAASSPSSNNTAATTAAIVPGSQLMPSKSPSTGTTEISSHESSHGTPSQTTAKNWELTASASHQPPGVYPQGHSDTTVAISTSTVLLCGLSAVSLLACYLKSRQTPPLASVEMEAMEALPVTWGTSSRDEDLENCSHHL

SEQ ID NO: 8 - DNA, 1266 bp, Artificial sequence
  IL-15-IL-15 RNA fusion protein amino acid sequence
atggtacttgggacaattgacctttgttcctgtttttcagccggtctgcctaaaactgaggcaaactgggtcaatgtgatatccgacctgaagaagatcgaagacttgatacagagcatgcatattgatgctaccctctacaccgaatccgacgtgcacccaagctgtaaggttactgctatgaaatgttttcttctggaacttcaggtcatctctcttgagtccggagacgcctccatccacgatactgttgagaatctgatcattttggctaataattcactgagcagcaacggtaatgtgaccgaaagcgggtgtaaagagtgcgaagagctggaaaagaagaatatcaaagagttcctccagtcttttgtacatatcgtacaaatgtttattaacacttcttcaggtggatctggagggggtggaagtggtggtggaagtggaggtggcggtagtggaggaatggctcctagaagggcacgagggtgcagaacactgggactgcctgccttgctcctgttgcttttgttgcgcccgccagcgacaaggggaatcacttgccctccccccatgtctgttgagcatgccgatatctgggtgaagtcttatagtctgtacagccgggaacggtacatttgcaatagcggcttcaaaagaaaagccggaactagttctctgactgaatgtgtcctgaacaaggccacaaacgtcgcacactggacaacgcccagtctgaaatgtattcgcgatcctgccttggttcaccagaggccagctcctccctcaactgtcaccactgctggcgtgactccacagccagagtcactgtcacccagcggtaaagagccagccgcatccagtccatcatctaacaacaccgctgctacaacagcagcaattgtgccaggatcacaactgatgccaagcaagtcaccatccaccggaacgacggaaatttcaagtcatgagtcctcccacggcacgccgagtcaaaccactgctaaaaattgggaactgactgcatctgcttcacaccagcctccgggtgtataccctcagggacactccgacacaactgtcgcaattagcacatctacagttctcttgtgcgggctttccgccgtgtctctgctcgcatgttatcttaaatctagacagacaccaccccttgcctcagtggaaatggaggccatggaagcactccccgttacatggggaacttccagccgcgacgaagatctggagaactgctcccaccacctg

SEQ ID NO: 9 - protein, 115 aa, Artificial sequence
  GC33 heavy chain protein amino acid sequence
QVQLQQSGAELVRPGASVKLSCKASGYTFTDYEMHWVKQTPVHGLKWIGALDPKTGDTAYSQKFKGKATLTADKSSSTAYMELRSLTSEDSAVYYCTRFYSYTYWGQGTLVTVSA

SEQ ID NO: 10 - DNA, 345 bp, Artificial sequence
  GC33 heavy chain protein DNA sequence
caagtgcaactgcaacaatccggtgccgaattggtaaggccaggtgctagcgtgaagctgagctgtaaggcaagcggctataccttcacggactatgaaatgcactgggtcaagcagacgccagtccatggcttgaagtggatcggcgcactcgacccaaagaccggggataccgcatactcccaaaaattcaaagggaaagccacactgaccgctgataagagtagctctactgcctatatggagttgagatctctcacctctgaggattccgcggtgtattactgcacacggttttacagctacacttactggggtcagggaactctcgttaccgtgtctgcc

SEQ ID NO: 11 - protein, 112 aa, Artificial sequence
  GC33 light chain protein amino acid sequence
DVVMTQTPLSLPVSLGDQASISCRSSQSLVHSNGNTYLHWYLQKPGGINSPKLLIYKVSNRFSGVPDRFSGSGSGTDFTLKISRVEAEDLGVYFCSQNTHVPPTFGSGTKLE

SEQ ID NO: 12 - DNA, 342 bp, Artificial sequence
  GC33 light chain DNA sequence
gatgtcgtgatgacgcaaacccctttgagcctgcctgtttcccttggggaccaggcttccatttcttgtaggtcttcccagtctctggttcactctaatggcaatacctacctccactggtatctccagaaacctggggggattaattcaccaaaattgctgatctacaaggtgagcaatcgcttctccggtgtcccagaccggttttcaggctctggttcagggaccgacttcacactcaagatttcacgggtagaggcagaggacctgggagtctacttctgctctcagaacacacatgtgcctccaacattcggctctgggaccaagctggaaataaag

SEQ ID NO: 13 - protein, 244 aa, Artificial sequence
  GC33 ScFv amino acid sequence
QVQLQQSGAELVRPGASVKLSCKASGYTFTDYEMHWVKQTPVHGLKWIGALDPKTGDTAYSQKFKGKATLTADKSSSTAYMELRSLTSEDSAVYYCTRFYSYTYWGQGTLVTVSAGGGGSGGGGSGGGGSDVVMTQTPLSLPVSLGDQASISCRSSQSLVHSNGNTYLHWYLQKPGGINSPKLLIYKVSNRFSGVPDRFSGSGSGTDFTLKISRVEAEDLGVYFCSQNTHVPPTFGSGTKLEIK

SEQ ID NO: 14 - DNA, 732 bp, Artificial sequence
  GC33 ScFv DNA sequence
caagtgcaactgcaacaatccggtgccgaattggtaaggccaggtgctagcgtgaagctgagctgtaaggcaagcggctataccttcacggactatgaaatgcactgggtcaagcagacgccagtccatggcttgaagtggatcggcgcactcgacccaaagaccggggataccgcatactcccaaaaattcaaagggaaagccacactgaccgctgataagagtagctctactgcctatatggagttgagatctctcacctctgaggattccgcggtgtattactgcacacggttttacagctacacttactggggtcagggaactctcgttaccgtgtctgccggcgggggcggatcaggcggcgggggatccggaggcggaggatccgatgtcgtgatgacgcaaacccctttgagcctgcctgtttcccttggggaccaggcttccatttcttgtaggtcttcccagtctctggttcactctaatggcaatacctacctccactggtatctccagaaacctggggggattaattcaccaaaattgctgatctacaaggtgagcaatcgcttctccggtgtcccagaccggttttcaggctctggttcagggaccgacttcacactcaagatttcacgggtagaggcagaggacctgggagtctacttctgctctcagaacacacatgtgcctccaacattcggctctgggaccaagctggaaataaag

SEQ ID NO: 15 - protein, 21 aa, Artificial sequence
  CD8 signal peptide amino acid sequence
MALPVTALLLPLALLLHAARP

SEQ ID NO: 16 - DNA, 60 bp, Artificial sequence
  CD8 signal peptide DNA sequence
atggcgcttcctgtcactgcacttttgctgccactggctttgcttcttcatgccgcaaga

SEQ ID NO: 17 - protein, 21 aa, Artificial sequence
  ICOS transmembrane region amino acid sequence
FWLPIGCAAFVVVCILGCILI

SEQ ID NO: 18 - DNA, 60 bp, Artificial sequence
  ICOS transmembrane region DNA sequence
ttctggcttccgattggctgcgctgcctttgtggtagtgtgcattctcgggtgcatactg

SEQ ID NO: 19 - protein, 21 aa, Artificial sequence
  CD8 transmembrane region amino acid sequence
IYIWAPLAGTCGVLLLSLVIT

SEQ ID NO: 20 - DNA, 60 bp, Artificial sequence
  CD8 transmembrane region DNA sequence
atttatatttgggctcccttggccggcacatgcggagttcttctgctgagcctggtcatt

SEQ ID NO: 21 - protein, 45 aa, Artificial sequence
  CD8 hinge region amino acid sequence
TTTPAPRPPTPAPTIASQPLSLRPEACRPAAGGAVHTRGLDFACD

SEQ ID NO: 22 - DNA, 135 bp, Artificial sequence
  CD8 hinge region DNA sequence
acaactacacctgcccctagacctcccacaccagcaccaacaattgcatctcagccgctgtcactgcgaccagaggcatgcagaccagccgctggtggcgctgttcataccagaggattggacttcgcatgcgat

SEQ ID NO: 23 - protein, 54 aa, Artificial sequence
  CD28 costimulatory domain amino acid sequence
RSKRSRLLHSDYMNMTPRRPGPTRKHYQPYAPPRDFAAYRSGAVHTRGLDFACD

SEQ ID NO: 24 - DNA, 123 bp, Artificial sequence
  CD28 costimulatory domain DNA sequence
cgcagcaaacggtctcgactgttgcactctgattatatgaatatgacaccccgaagacctggccccacaagaaagcactatcagccatacgctcccccaagagactttgccgcttataggtcc

SEQ ID NO: 25 - protein, 15 aa, Artificial sequence
  linker amino acid sequence
GGGGSGGGGSGGGGS

SEQ ID NO: 26 - DNA, 45 bp, Artificial sequence
  linker DNA sequence
ggcgggggcggatcaggcggcgggggatccggaggcggaggatcc

SEQ ID NO: 27 - protein, 42 aa, Artificial sequence
  4-1BB costimulatory domain amino acid sequence
KRGRKKLLYIFKQPFMRPVQTTQEEDGCSCRFPEEEEGGCEL

SEQ ID NO: 28 - DNA, 126 bp, Artificial sequence
  4-1BB costimulatory domain DNA sequence
aaacgaggcagaaagaaactcctgtatatcttcaagcagccctttatgagaccagtccaaaccacccaggaggaagacggctgctcttgtaggttcccggaggaagaagagggtggatgcgaactg

SEQ ID NO: 29 - protein, 8 aa, Artificial sequence
  ICD1 amino acid sequence
VQPEEEED

SEQ ID NO: 30 - DNA, 24 bp, Artificial sequence
  ICD1 DNA sequence
gttcaacctgaggaagaagaagat

SEQ ID NO: 31 - protein, 8 aa, Artificial sequence
  ICD2 amino acid sequence
RFTTQEEG

SEQ ID NO: 32 - DNA, 24 bp, Artificial sequence
  ICD2 DNA sequence
agattcacaacccaggaggaggga

SEQ ID NO: 33 - protein, 8 aa, Artificial sequence
  ICD3 amino acid sequence
RTPEEEEQ

SEQ ID NO: 34 - DNA, 24 bp, Artificial sequence
  ICD3 DNA sequence
cgaacccccgaagaggaggagcag

SEQ ID NO: 35 - protein, 113 aa, Artificial sequence
  CD3 intracellular signal domain amino acid sequence
RVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 36 - DNA, 339 bp, Artificial sequence
  CD3 intracellular signal domain DNA sequence
agggtaaaattctccagaagtgcagatgcccccgcttatcagcagggccaaaatcagctctataatgaactgaatcttgggcgccgcgaggaatacgatgtgctcgacaaacgacggggtcgcgatccagaaatgggaggcaagcctcagaggcgcaagaacccgcaggaaggactctacaatgagttgcagaaggacaagatggcagaagcatacagtgagattgggatgaaaggcgaaaggaggaggggaaaggggcatgatgggttgtatcagggcttgtctactgctacaaaggacacgtatgacgcactccacatgcaggctctgcccccaaga

SEQ ID NO: 37 - DNA, 323 bp, Artificial sequence
  pGK300 promoter DNA sequence
acccctctccagccactaagccagttgctccctcggctgacggctgcacgcgaggcctccgaacgtcttacgccttgtggcgcgcccgtccttgtcccggggtgtgatggcggggtgtggggcggagggcgtggcggggaagggccggcgacgagagccgcgcgggacgactcgtcggcgataaccggtgtcgggtagcgccagccgcgcgacggtaacgagggaccgcgacaggcagacgctcccatgatcactctgcacgccgaaggcaaatagtgcaggccgtgcggcgcttggcgttccttggaagggctgaatccccg

SEQ ID NO: 38 - protein, 18 aa, Artificial sequence
  T2A amino acid sequence
EGRGSLLTCGDVEENPGP

SEQ ID NO: 39 - DNA, 54 bp, Artificial sequence
  T2A DNA sequence
gagggcagaggaagtctgctaacatgcggtgacgtcgaggagaatcctggccca

SEQ ID NO: 40 - DNA, 6217 bp, Artificial sequence
  pLV300 vector DNA sequence
acgcgtgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattgccgcattgcagagatattgtatttaagtgcctagctcgatacaataaacgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacctgaaagcgaaagggaaaccagagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccactgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcctcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagattggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcggttaacttttaaaagaaaaggggggattggggggtacagtgcaggggaaagaatagtagacataatagcaacagacatacaaactaaagaattacaaaaacaaattacaaaattcaaaattttatcgatagatacgaattaattctaccacccctctccagccactaagccagttgctccctcggctgacggctgcacgcgaggcctccgaacgtcttacgccttgtggcgcgcccgtccttgtcccggggtgtgatggcggggtgtggggcggagggcgtggcggggaagggccggcgacgagagccgcgcgggacgactcgtcggcgataaccggtgtcgggtagcgccagccgcgcgacggtaacgagggaccgcgacaggcagacgctcccatgatcactctgcacgccgaaggcaaatagtgcaggccgtgcggcgcttggcgttccttggaagggctgaatccccgacggatccgctagcgtttaaacgcggccgcgaattcgtcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggtacctttaagaccaatgacttacaaggcagctgtagatcttagccactttttaaaagaaaaggggggactggaagggctaattcactcccaacgaaaataagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccagttccgcccattctccgccccatggctgactaattttttttatttatgcagaggccgaggccgcctcggcctctgagctattccagaagtagtgaggaggcttttttggaggcctagacttttgcagagacggcccaaattcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgccaagctg

(19) United States
(12) Patent Application Publication
(10) Pub. No.: US 2024/0415965 A1
(43) Pub. Date: Dec. 19, 2024

(54) CAR-INKT WITH HIGH AMPLIFICATION, SURVIVAL CAPACITY AND TUMOR KILLING EFFECT AND USE THEREOF

(71) Applicant: Beijing Gene Key Life Technology Co., Ltd., Beijing (CN)

(72) Inventors: Jun LYU, Beijing (CN); Fuyin XIONG, Beijing (CN); Jikai FENG, Beijing (CN)

(21) Appl. No.: 18/266,993

(22) PCT Filed: Oct. 22, 2021
(86) PCT No.: PCT/CN2021/125615

§ 371 Date: Jun. 13, 2023

(30) Foreign Application Priority Data
Dec. 14, 2020 (CN) 202011461635.3

(51) Int. Cl. A61K 39/00; A61P 35/00; C07K 14/54; C07K 14/725; C07K 16/30; C12N 5/0783

(52) U.S. Cl. CPC A61K 39/4631; A61K 39/4611; A61K 39/4613; A61K 39/4635; A61P 35/00; C07K 14/5443; C07K 14/7051; C07K 16/303; C12N 5/0636; C12N 5/0646; C07K 2317/622; C07K 2319/02; C07K 2319/30

(57) ABSTRACT

Provided in the present application is a chimeric antigen receptor, including a GPC3 antigen binding domain, an ICDI, ICD2 or ICD3 intracellular signal stimulation domain with amino acid sequences of SEQ ID NOs: 29, 31 and 33, respectively, and an IL-15-IL-15α fusion protein with an amino acid sequence of SEQ ID NO: 7. After the chimeric antigen receptor is transferred into immune cells, especially iNKT cells, the cell proliferation rate, survival time and tumor killing effect can be effectively improved. Further provided in the present application are a corresponding expression vector, a transduction system, a pharmaceutical use, independent ICDI, ICD2 and ICD3 intracellular signal stimulation domains, and an IL-15-IL-15α fusion protein.

### CROSS-REFERENCE TO RELATED APPLICATIONS

This application is a National Stage of International Application No. PCT/CN2021/125615, filed Oct. 22, 2021, which in turn claims the benefit of Chinese Patent Application 202011461635.3, filed Dec. 14, 2020. The entire disclosures of the above applications are incorporated herein by reference.

### TECHNICAL FIELD

The present application belongs to the field of cancer immunotherapy. In particular, the present application provides a chimeric antigen receptor, which comprises a GPC3 antigen binding domain, an intracellular signal stimulation domain and an IL-15-IL-15α fusion protein; the corresponding expression vectors, transduction systems and pharmaceutical use are also provided.

### BACKGROUND

According to the 2018 Global Cancer statistics, there are 841,080 new cases of liver cancer worldwide, and 781,631 people died of liver cancer [1]. Among them, there are 392,868 new cases of liver cancer in China, and 368,960 people died of liver cancer [2], accounting for almost half of all new cases and deaths of liver cancer in the world.

Current treatment methods for liver cancer comprise surgical resection, liver transplantation, local treatment (RFA, TACE, TAE and HIFA, etc.), and systematic treatment (sorafenib and lenvatinib, etc.). Surgical resection can achieve a cure, but most liver cancers are detected when the optimal timing of surgery is missed, so the treatment for liver cancer is not satisfactory. Studies have shown that the median survival time for liver cancer patients is only 23 months in China, 60 months in Japan, 33 months in North America, 31 months in Korea, 24 months in Europe, 11 months in Egypt, and only 3 months in other African countries [3]. There is an urgent need for a more safe and effective treatment for liver cancer.

Chimeric antigen receptor technology has achieved significant efficacy in the treatment of hematological tumors. Kymarih [4] and Yescarta [5] were approved by FDA in 2017 for the treatment of B-cell acute lymphoblastic leukemia and diffuse large B-lymphoma. GPC3 is highly expressed in 70% of hepatocellular carcinoma cells, but not in normal adult tissues [6]. GPC3 is also overexpressed in hepatoblastoma, squamous cell lung cancer, testicular and ovarian yolk sac tumors, melanoma, ovarian clear cell carcinoma and other tumors [7], which is an ideal target for targeted therapy of chimeric antigen receptor technology.

GPC3 gene is located on X chromosome, has 11 exons, a transcript of 2130 bp, encodes 580 amino acids, and its molecular weight is about 70 kDa. The GPC3 polypeptide contains a Furin restriction site that cleaves the polypeptide between Arg358 and Cys359 into two fragments: 40 kDa at the N-terminus and 30 kDa at the C-terminus. These two subunits can be linked by one or more disulfide bonds. N-terminal subunits can be further cleaved to form soluble GPC3 in peripheral blood circulation. GPC3 can be modified with heparin sulfate in Cys495 and Cys508. GPC3 Ser560 is anchored to the lipid layer of the cell membrane by phosphatidylinositol [8].

Under normal physiological conditions, GPC3 is widely expressed on different embryonic cell membranes, but not in normal adult liver tissues. Mutations in human GPC3 cause SGBS (simpson golabi behmel syndrome), which manifests as macrosomia accompanied by multiple organ and bone dysplasia. GPC3 is anchored on the cell membrane and has no intracellular region, but it can interact with different growth factors, chemokines and cytokines to form a concentration gradient on the surface of the cell membrane, thereby promoting the binding of these ligands to their receptors [8].

Invariant natural killer T cell (iNKT) is a unique subset of thymus-derived T cells with CD1d restriction, and simultaneously expresses surface receptors with lineage characteristics of T cells and natural killer cells (NK). It has the common biological characteristics of T cells and NK cells and plays an important role in bridging innate and adoptive immunity. In human iNKT cells, Vα 24-Jα 18 forms TCR α chain and then forms TCR with Vβ11 TCRβ chain [9].

INKT cells differentiate into at least three effector subsets in the thymus, similar to the subset of CD4+ T helper cells and can also resemble the subset of innate lymphocytes (ILCs) [10-12]. Functional iNKT cell subsets are distinguished according to the expression of different cell surface markers and characteristic transcription factors. NKT1 cells are similar to Th1 cells and ILC1s because they both highly express the transcription factor T-bet and secrete IFN-γ after activation. NKT1 cells also showed greater cytotoxicity than other iNKT cell subsets. NKT1 cells differ from Th1 cells or ILC1s in that produce factors such as IL-4 in addition to IFN-γ through TCR activation. The cytokines secreted by NKT2 cells comprise IL-4 and IL-13, which are similar to Th2 cells. NKT17 cells are similar to Th17 cells in cytokine secretion [13-15].

Different iNKT cell subsets are enriched in different tissues. NKT1 cells are highly enriched in the liver, while NKT17 cells are mainly located in lymph nodes, skin and lungs, and a few cells are found in the spleen [16]. NKT2 cells are located in many sites, including lung and spleen, but they are particularly abundant in mesenteric lymph nodes [16]. In peripheral lymph nodes, iNKT cells can be rapidly activated and may play a key role in fighting pathogens [17].

INKT cells in human blood can be divided into DN iNKT (double negative, DN) cells, CD4+ iNKT cells and CD8+ iNKT (CD8 αα or CD8 αβ) [9]. In particular, CD8+ iNKT cells are only found in humans. Studies have shown that when DN iNKT and CD8+iNKT cells are activated, their IFN-γ secretory and cytotoxic functions were significantly increased [9].

The current GPC3 chimeric antigen receptors, such as those in U.S. Pat. No. 10,731,127B2 and CN 109468279 A, which are closed to the present patent, enable T cells to express chimeric receptors targeting GPC3 antigen through gene modification, wherein CAR comprising a GPC3 antigen binding domain, a transmembrane domain, a costimulatory signal transduction region and a CD3 ζ signal transduction domain, and show a killing effect on hepatoma cells carrying GPC3 antigen. However, in view of the fact that conventional T cells cannot effectively infiltrate into solid tumors; the tumor microenvironment of solid tumors is anoxic and acidic, which is very unfavorable to the expansion and long-term survival of CAR-T cells, thus seriously affecting the efficacy of CAR-T cells.

### SUMMARY OF THE INVENTION

In order to solve the above problems, the applicant uses the homing property and non-specific killing function of iNKT cells to make them carry chimeric antigen receptors that can bind GPC3 antigen via gene modification, and constructs anti-GPC3-CAR−iNKT cells that can specifically kill hepatoma cells carrying GPC3 antigen.

The gene modification enables anti-GPC3-CAR−iNKT cells to specifically express cytokines that promote the differentiation of anti-GPC3-CAR−iNKT cells into CD8⁺ anti-GPC3-CAR−iNKT cells and CD4⁻ CD8⁻ anti-GPC3-CAR−iNKT cells upon activation, thus enhances the specific and non-specific killing effect of anti-GPC3-CAR−iNKT cells on hepatoma cells.

By optimizing the combination of co-stimulatory domains, the expansion and long-term survival of anti-GPC3-CAR−iNKT cells are improved, the apoptosis of anti-GPC3-CAR−iNKT cells is reduced, and the anti-tumor function of anti-GPC3-CAR−iNKT cells in patients is fully utilized.

TRAF family molecules can contribute to cell proliferation and anti-apoptosis by binding to downstream molecules such as RIP and TRADD, which ultimately activate the NF-κB pathway. By drawing on the motif combined with TRAF family molecules, we design three intracellular signal domains, ICD1, ICD2 and ICD3, which are constructed into CAR molecules for observing the changes on CAR−iNKT cell proliferation, cell subset and ability to kill tumor cells when the intracellular signaling domains are ICD1, ICD2 and ICD3, respectively.

IL-15 can induce T cell proliferation and differentiation to CD8+ T cell subsets, thereby increasing the cytotoxic effects of T cells. However, when IL-15 acts, it must bind to IL-15Rα before it can bind to IL-15Rγ, thereby stimulating cell proliferation and differentiation signals. In the present application, constructing the co-expression of IL-15-IL-15Rα fusion protein in the CAR molecule can promote the differentiation of CAR−iNKT cells into CD8+ CAR−iNKT cell subsets and promote CAR−iNKT cells proliferation, so as to be more conducive to CAR−iNKT killing tumor cells, and meet the needs of clinical application transformation.

Compared with the prior art, a technical solution of the present application has improved tumor killing efficiency and accelerated proliferation of CAR−iNKT cells, which can shorten the cell culture cycle in vitro and increase the survival time of CAR−iNKT cells in vivo, greatly improve the efficacy, reduce recurrence and mitigate toxic side effects.

In an aspect, the present application provides a chimeric antigen receptor, comprising a GPC3 antigen binding domain and an intracellular signal stimulation domain.

Further, the chimeric antigen receptor further comprises an IL-15-IL-15α fusion protein.

Further, the intracellular signal stimulation domain is ICD1, ICD2 or ICD3 with the amino acid sequences SEQ ID Nos. 29, 31 and 33 respectively.

Further, the intracellular signal stimulation domain is ICD3 with the amino acid sequence SEQ ID NO.33.

Further, the amino acid sequence of the IL-15-IL-15α fusion protein is SEQ ID NO. 7.

Further, the GPC3 antigen binding domain is GC33 ScFv containing amino acid sequences SEQ ID NOs. 9 and 11.

Further, the GPC3 antigen binding domain is GC33 ScFv with the amino acid sequence SEQ ID NO.13.

Further, the chimeric antigen receptor comprises sequentially connected GC33 ScFv, a hinge region, a transmembrane domain, a costimulatory signal domain, ICD1 or ICD2 or ICD3, a CD3 ζ signal conduction domain and a IL-15-IL-15α fusion protein.

Further, the amino acid sequences of the hinge region, the transmembrane domain, the costimulatory signal domain, and the CD3 ζ signal conduction domain are SEQ ID NOs.21, 19, 23 and 35, respectively.

In another aspect, the present application provides an immune cell, wherein the immune cell is transduced into the chimeric antigen receptor.

Further, the immune cell is T cell, NK cell or iNKT cell.

Further, the immune cell is iNKT cell.

In another aspect, the present application provides use of the chimeric antigen receptor or the immune cells in the manufacture of a medicament for treating cancer.

In another aspect, the present application provides a method for treating cancer, wherein the chimeric antigen receptor or the immune cells is used.

Further, the cancer is a cancer with overexpression of GPC3.

Further, the cancer is liver cancer.

In another aspect, the present application provides an expression vector of the chimeric antigen receptor.

Further, the expression vector comprises ICD1, ICD2 or ICD3 nucleic acid sequences with the nucleotide sequences SEQ ID Nos. 30, 32 and 34 respectively.

Further, the expression vector comprises IL-15-IL-15α fusion protein nucleic acid sequence with the nucleotide sequence SEQ ID NO.8.

In another aspect, the present application provides a transduction system comprising the above-described expression vector.

Further, the transduction system is a viral transduction system and a non-viral transduction system.

Further, the transduction system is a lentiviral transduction system.

In another aspect, the present application provides an intracellular signal stimulatory molecule, wherein the amino acid sequence is SEQ ID NO. 29, 31 or 33, respectively.

In another aspect, the present application provides the nucleic acid coding sequence of the above-mentioned intracellular signal stimulatory molecule, and the nucleic acid coding sequence is SEQ ID NO. 30, 32 or 34.

In another aspect, the present application provides an IL-15-IL-15α fusion protein, and the amino acid sequence is SEQ ID NO.7.

In another aspect, the present application provides the nucleic acid coding sequence of the above-mentioned IL-15-IL-15α fusion protein, and the nucleic acid coding sequence is SEQ ID NO.8.

In another aspect, the present application provides use of the intracellular signal stimulatory molecule or nucleic acid coding sequence thereof, or the IL-15-IL-15α fusion protein or nucleic acid coding sequence thereof in the manufacture of a chimeric antigen receptor for treating cancer.

The types of cancers with overexpression of GPC3 in present application are known to those skilled in the art from prior art before the application date and subsequent research.

The optimized and modified intracellular signal stimulation domain of present application, ICD1, ICD2 and ICD3 as examples in present application, can accelerate the proliferation of CAR−iNKT and promote CAR−iNKT to secrete more IFN-γ, enhance the tumor-killing efficacy of CAR−iNKT cells, reduce the depletion of CAR−iNKT cells and prolong the survival time of CAR−iNKT in vivo.

The IL-15-IL-15Rα fusion protein nucleic acid of the present application can induce an increase in the proportion of CD8+ CAR+ iNKT cells, promote CAR−iNKT to secrete more IFN-γ, enhance the tumor-killing efficacy of CAR−iNKT cells, reduce the depletion state of CAR−iNKT cells, and prolong the survival time of CAR−iNKT in vivo.

The present application comprises a nucleic acid expression vector comprising one or more of the above elements for simultaneously constructing chimeric antigen receptor protein expressed on the surface of T cells, NK cells or NKT cells. Various commercially available vectors can be selected according to needs, or vectors can be constructed according to routine technology in the art of molecular biology. In one specific embodiment, the vector used in present application is a lentivirus plasmid vector pLV300. The plasmid belongs to the fourth generation of self-inactivating lentivirus vector system. The system has four plasmids, namely a packaging plasmid encoding Gag/Pol protein, a packaging plasmid encoding Rev protein, an envelope plasmid encoding VSV-G protein, and an empty vector pLV300, which can be used for recombinantly introducing the target nucleic acid, namely, the nucleic acid sequence encoding chimeric antigen receptor protein. The expression of chimeric antigen receptor protein is regulated by pGK-300 promoter in vector pLV300.

The application comprises viruses comprising the above vectors, comprising but not limited to lentivirus, retrovirus, adenovirus, adeno-associated virus, etc. The viruses of the present application comprise viruses that are packaged to be infectious, and also comprise viruses to be packaged that contain the components necessary for packaging as an infectious virus. Other viruses known in the art for transfecting T cells, NK cells or NKT cells and their corresponding plasmid vectors can also be used in the present application. In one embodiment of the present application, the virus is a lentivirus comprising the pLV300-anti-GPC3 CAR recombinant vector.

The present application comprises a transgenic T lymphocyte, NK cell or iNKT cell, which is transduced with the nucleic acid of present application or the above-mentioned recombinant plasmid comprising the nucleic acid of present application, or a virus system comprising the plasmid. Routine nucleic acid transduction methods in the art, comprising non-viral and viral transduction methods, can be used in present application. Non-viral transduction methods comprise electroporation and transposon. The nucleofector recently developed by Amaxa Company is able to directly introduce exogenous genes into the nucleus of cells to obtain efficient transduction of target genes. In addition, the transduction efficiency of transposon systems such as Sleeping Beauty system or PiggyBac transposon has been improved compared with ordinary electroporation, and the combination of nucleofector and SB Sleeping Beauty transposon system has been reported to achieve both high transduction efficiency and targeted integration of target genes. In one embodiment of the present application, the transduction method for achieving chimeric antigen receptor gene-modified iNKT cells is a transduction method based on lentivirus. This method has the advantages of high transduction efficiency, stable expression of exogenous genes, and shortening the time to reach clinical grade numbers of iNKT lymphocytes cultured in vitro. The nucleic acid transfected by lentivirus is expressed on the surface of iNKT cell membrane via transcription and translation. Through in vitro cytotoxicity tests on various cultured tumor cells, it is proved that the transgenic iNKT cells expressing chimeric antigen receptors on the surface of the present application have highly specific tumor cell killing effects (also known as cytotoxicity). Therefore, the nucleic acid encoding the chimeric antigen receptor protein, the plasmid containing the nucleic acid, the virus containing the plasmid and the transgenic iNKT cells, T lymphocytes or NK cells transfected with the nucleic acid, the plasmid or virus can be effectively used for tumor immunotherapy.

When the chimeric antigen receptor protein is constructed by linking the intracellular signal domain and the extracellular target-binding region in the application, the extracellular-binding region comprises receptors or ligands that specifically recognize corresponding targets, including but not limited to existing known targets, specific single chain antibodies scFv, TCRm, and VLR. Human GPC3 target is taken as an example for exploration in the application. The receptor or ligand, specific single chain antibody scFv, TCRm or VLR can be prepared by genetic engineering methods or chemical synthesis methods according to the sequences disclosed in the literature above.

The nucleic acids of the present application may be in the form of DNA or RNA. DNA forms include cDNA, genomic DNA or synthetic DNA. DNA may be single-stranded or double-stranded. The DNA may be a coding or non-coding strand. The nucleic acid codons encoding the amino acid sequences of the chimeric antigen receptor proteins of the present application may be degenerate, i.e., multiple degenerate nucleic acid sequences encoding the same amino acid sequence are within the scope of present application. The degenerate nucleic acid codons encoding the corresponding amino acids are well known in the art. The application also relates to variants of the above polynucleotides, which encode polypeptides or fragments, analogues and derivatives of polypeptides having the same amino acid sequence as the present application. The variants of this polynucleotide can be naturally occurring allelic variants or non-naturally occurring variants. These nucleotide variants comprise substitution variants, deletion variants, and insertion variants. As is known in the art, an allelic variant is a substitution form of a polynucleotide that may be a substitution, deletion, or insertion of one or more nucleotides without substantially altering the function of the polypeptide it encodes.

In the existing CAR-T and CAR-NK technologies for the treatment of solid tumors, the effect of CAR-T and CAR-NK in the treatment of solid tumors is limited because conventional T cells and NK cells cannot effectively infiltrate into solid tumors, and NK cells are limited to expand in vitro. In the present application, the optimization of the intracellular signal domain can promote the effective amplification of CAR iNKT, reduce the depletion of CAR iNKT, and prolong the survival time of CAR iNKT in vivo. At the same time, with co-expression of IL-15-IL-15Rα, CAR iNKT differentiates into CD8+CAR iNKT and CD4−CD8−CAR−iNKT phenotypes that are more conducive to killing tumors, and enhances the killing effect of CAR iNKT on tumor cells. This is an advantage that current CAR-T and CAR-NK technologies for the treatment of solid tumors do not have. The combination of intracellular signaling domains and co-expression of IL-15-IL-15Rα fusion protein in the present application provides the possibility of CAR−iNKT technology for the treatment of solid tumors.

### BRIEF DESCRIPTION OF THE DRAWINGS

FIG. 1 shows the curve of total iNKT cells proliferation;

FIG. 2A shows the proportion of CD8+ CAR+ iNKT cell subsets at different times of CAR−iNKT cell culture;

FIG. 2B shows the proportion of CD8+ CAR+ iNKT cell subsets at different times of CAR−iNKT cell culture;

FIG. 2C shows the proportion of CD4+ CAR+ iNKT cell subsets at different times of CAR−iNKT cell culture;

FIG. 2D shows the proportion of CD4−CD8−CAR+iNKT cell subsets at different times of CAR−iNKT cell culture;

FIG. 3A shows the killing effect of anti-GPC3 CAR−iNKT on hepatoma cells Huh-7 at different effector-target ratios;

FIG. 3B shows the multiple of enhancement of the killing effect of anti-GPC3 CAR−iNKT on hepatoma cells Huh-7 in each group at different effector-target ratios compared with anti-GPC3 CAR−iNKT with intracellular signal domain of 4-1BB;

FIG. 4A shows the IFN-γ content in cell culture supernatant after 24 hours of co-culture of anti-GPC3 CAR−iNKT cells with hepatoma cells;

FIG. 4B shows the multiple of change in IFN-γ content in cell culture supernatant after 3:1 inoculation of each group of anti-GPC3 CAR−iNKT cells with hepatoma cells Huh-7 and co-culture for 24 hours compared to the anti-GPC3-CAR−iNKT group with an intracellular signal domain of 4-1BB;

FIG. 5 shows the expression of LAG-3 after 24 hours of co-culture of anti-GPC3 CAR−iNKT cells with hepatoma cells;

FIG. 6 shows the structure diagram of anti-GPC3 CAR; and

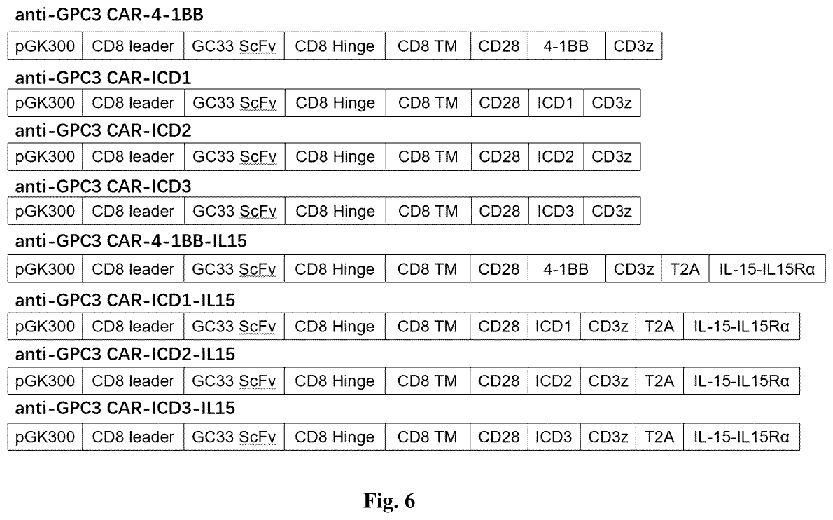

FIG. 7 shows the structure diagram of the structure of lentiviral vector.

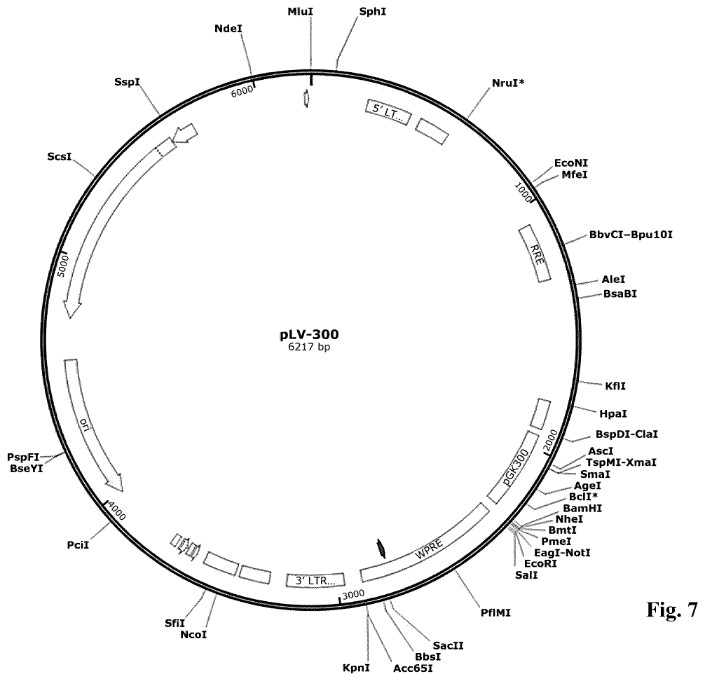

### DETAILED DESCRIPTION

### Terms and Abbreviations

| Abbreviation | English Full Name |
|---|---|
| CAR | Chimeric Antigen Receptor |
| CAR-T | Chimeric Antigen Receptor-T cell |
| CAR-iNKT | Chimeric Antigen Receptor-iNKT |
| | cell |
| CAR-NK | Chimeric Antigen Receptor-NK cell |
| TCR | T cell Receptor |
| IFN-γ | interferon-γ |
| IL-2 | Interleukin-2 |
| IL-15 | Interleukin-15 |
| IL-15Rα | Interleukin-15 Receptor α |
| ICD | Intracytoplasmic signal domain |
| scFv | single-chain fragment variable |
| pGK | phosphoglycerate kinase |
| α-GalCer | α-galactosylceramide |

### Example 1: Preparation of Lentivirus Containing Chimeric Antigen Receptor Molecule

HEK-293T cells were passaged, and after the cells growth to reach 60%-70% confluency, the expression vector containing CAR molecules was transfected into HEK-293T cells together with the packaging plasmid by using pEI reagent, and the medium was replaced with fresh medium 4 hours after transfection. Cell culture supernatant was collected 48-72 hours after transfection. The supernatant was ultracentrifuged to concentrate the lentivirus containing CAR molecules after packaging. The virus titer of the concentrated lentivirus was determined and stored at −80° C. for future use.

The specific nucleotide and amino acid sequences of each part of the chimeric antigen receptor are shown in the sequence list, in which the names of each part have been marked.

### Example 2: The Effect of Different Intracellular Signal Stimulation Domains Construction in Anti-GPC3 CAR and Whether or not Expressing IL-15-IL15Rα Fusion Protein on the Proliferation of Total iNKT Cells

After iNKT cells were isolated from human peripheral blood mononuclear cells with anti iNKT microbeads, they were inoculated with 2×10⁵ cells per well in a 24-well plate, and X-VIVO complete medium containing 100 IU/ml and 100 ng/ml α-Galcer was added to each well and culturing 48 hours, then the cells from each well were collected, and centrifugated at 400×g for 5 minutes, the supernatant was discarded. Fresh X-VIVO complete culture medium was added for resuspending, and then reinoculated them into a 24-well culture plate. Each well cell was infected by different lentiviruses containing 4-1BB, ICD1, ICD2, ICD3, 4-1BB-IL-15, ICD1-IL-15 and ICD3-IL-15 CAR. After 24 hours, cells of each well were collected into a centrifuge tube, and counted after centrifugated with 400×g for 5 minutes. Fresh X-VIVO complete medium was added at 5×10⁵ cells/ml for culture, and the solution was changed every 48 hours. When the cells were cultured to 7th day, 14th day and 21st day respectively, the samples were counted and flow cytometry was performed to prove the expression of chimeric antigen receptor molecules.

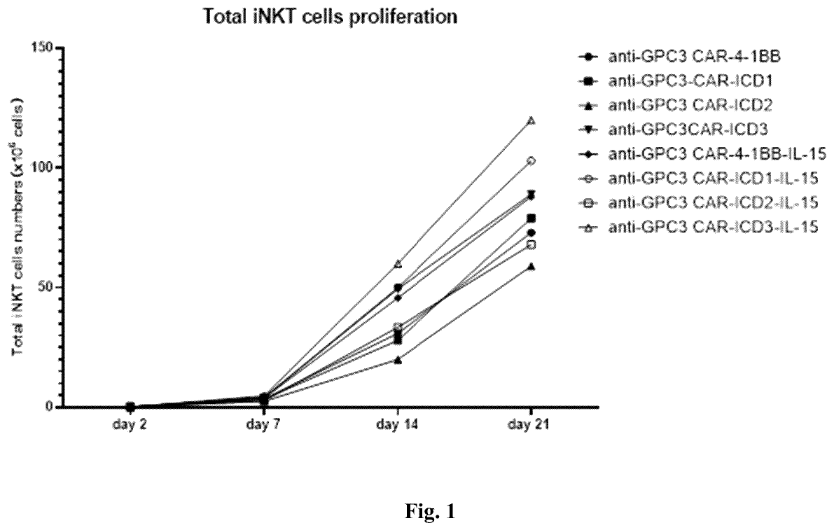

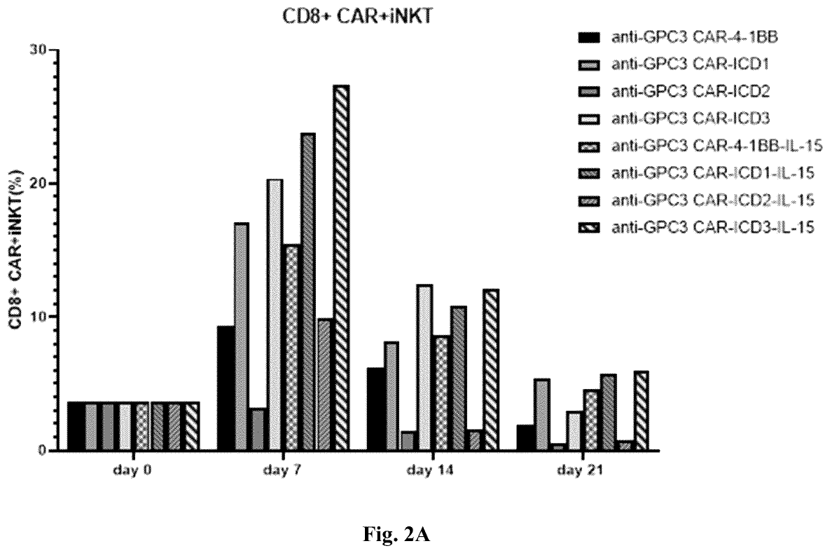

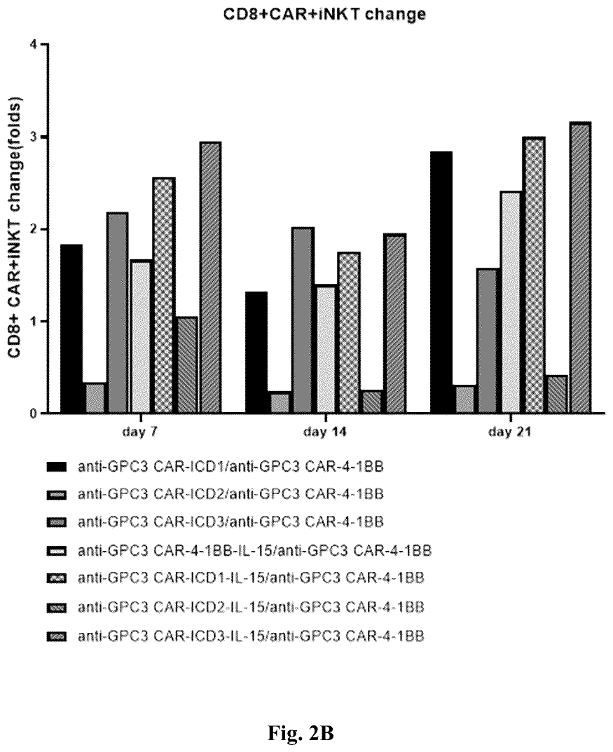

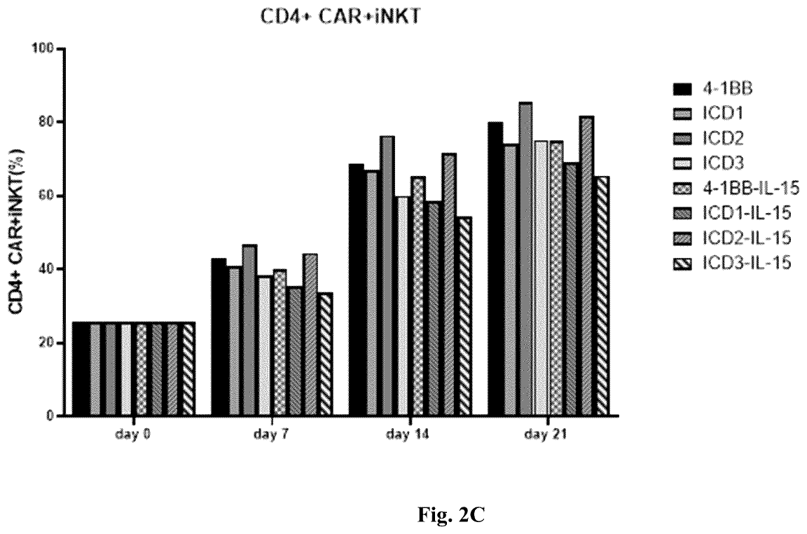

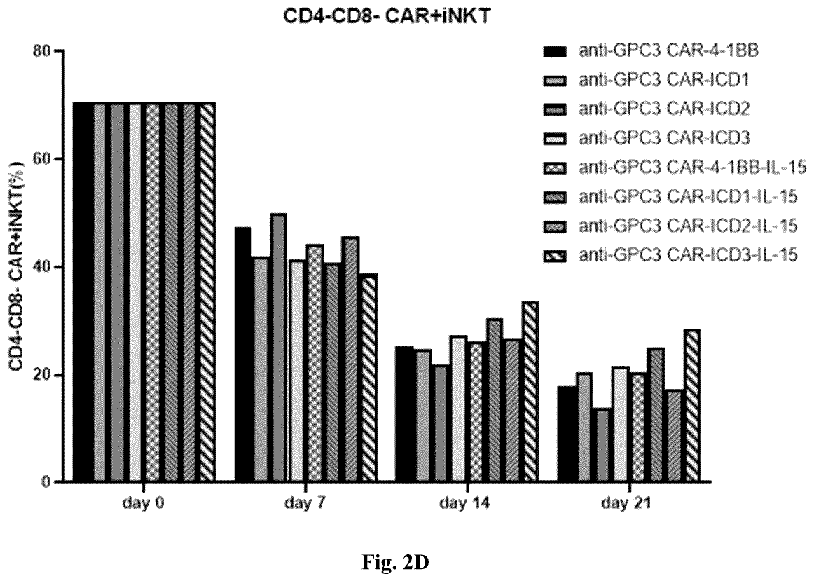

The results showed that ICD1, ICD3 and IL-15-IL-15α fusion protein can obviously promote the proliferation of iNKT cells (see FIG. 1) and increase the number of CD8+CAR+iNKT cells. On the 7th day of culture, the proportion of CD8+CAR+iNKT cells in CAR iNKT cells in each group is the highest, and the proportion of CD8+CAR+iNKT cells in CAR iNKT group with ICD3 as the signal domain in cells can reach 20%-30% (coexpressing of IL-15-IL-15Rα or not expressing IL-15-IL-15Rα). Later, with the extension of culture time, the proportion of CD8+CAR+iNKT cells gradually decreased, but compared with CAR iNKT cells with 4-1BB intracellular signal stimulation domain, CAR iNKT cells of ICD1 and ICD3 intracellular signal stimulation domains increased 2-3 times in the same period (FIG. 2A and FIG. 2B).

During the culture process, the proportion of CD4+CAR+iNKT cells in each group gradually increased (FIG. 2C), but compared with CAR iNKT cells with 4-1BB intracellular signal stimulation domain, the proportion of CD4+CAR+iNKT cells of ICD1 and ICD3 intracellular signal stimulation domains increased less. The proportion of CD4−CD8−CAR+iNKT cells in cells of each group has been decreasing with the culture time. In the CAR iNKT group with ICD3 as the intracellular signal domain, the proportion of CD4−CD8−CAR+iNKT cells is the lowest among the CAR iNKT cells of each group at the same time point (FIG. 2D).

### Example 3: The Effect of Different Intracellular Signal Domains and Whether Expressing Fusion Protein IL-15-IL-15Rα on Tumor Killing Ability of CAR iNKT

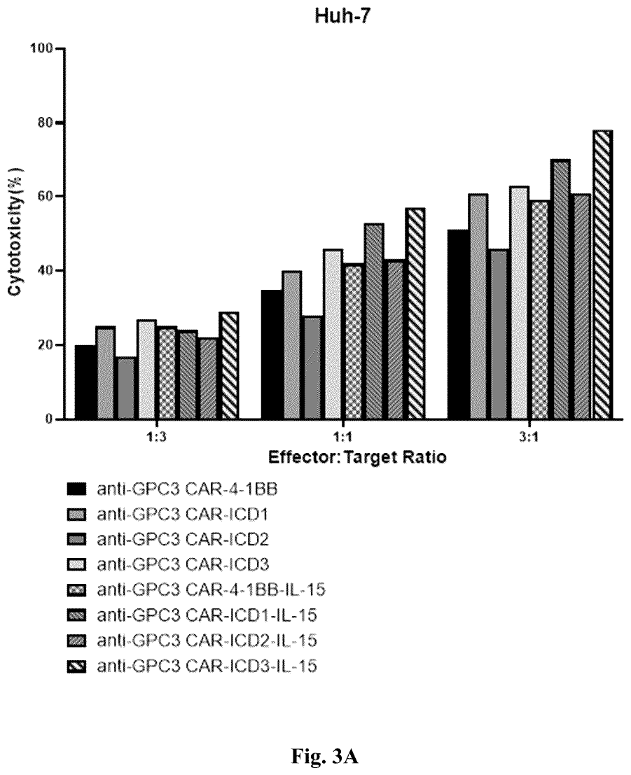

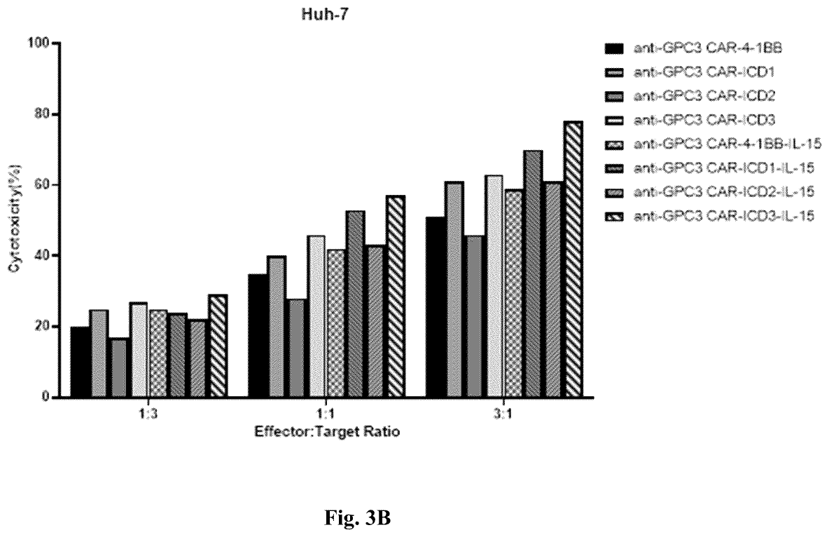

When CAR iNKT cells of different groups were cultured to the 21st day, 0.33×10⁵ CAR iNKT cells, 1×10⁵ CAR iNKT cells, 3×10⁵ CAR iNKT cells, and 1×10⁵ Huh-7 cells (mixed and incubated with fluorescent dye CFSE for 10 minutes) were taken from each group according to the ratio of effector cells to target cells of 1:3, 1:1, and 3:1, respectively. The culture supernatant of each group was taken and the fluorescence intensity in the supernatant was measured with the microplate reader (the stronger the killing capacity of CAR iNKT cells, the more CFSE released from Huh-7 cells, the greater the fluorescence intensity), and the killing efficiency of CAR iNKT cells in each group were calculated (FIG. 3). CAR−iNKT cells with ICD1 and ICD3 as intracellular signal domains have significantly enhanced the killing effect on hepatoma cells Huh-7, and co express IL-15-IL-15Rα will significantly increase the killing efficiency of CAR−iNKT cells with ICD1 and ICD3 signal domains (FIG. 3A, FIG. 3B).

### Example 4: The Effect of Different Intracellular Signal Domains and Whether Expressing Fusion Protein IL-15-IL-15Rα CAR iNKT Cells Secrete IFN-γ

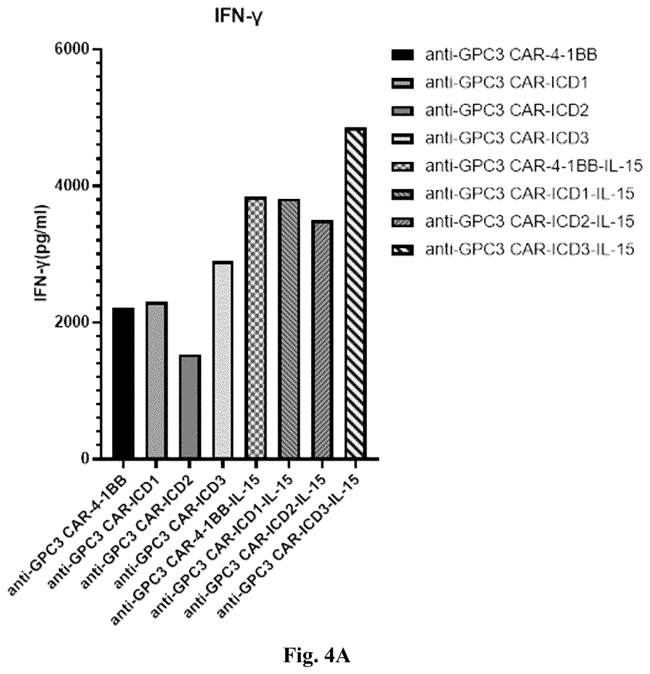

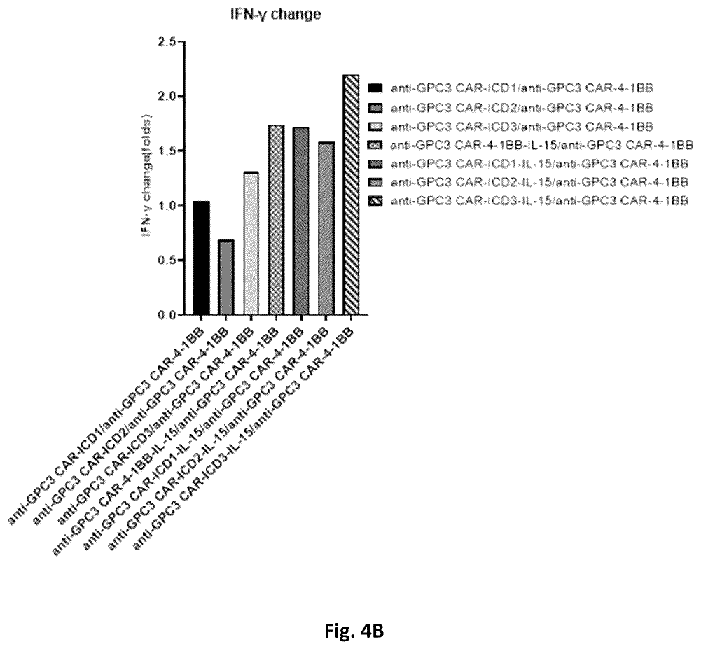

When CAR iNKT cells in each group were cultured to the 21st day, CAR iNKT cells in each group (3×10⁵ CAR−iNKT cells in each group) and 1×10⁵ Huh-7 cells were taken according to the ratio of effector cells to target cells 3:1 and co cultured in 0.5 ml X-VIVO (without IL-2 and α-GalCer) for 24 hours, the cells were collected and centrifugated at 400×g for 5 minutes, then the supernatant was taken, and the content of IFN-γ in the supernatant was detect by ELISA. It can be seen that when the intracellular signal domain is ICD1 or ICD3, it can significantly increase the secretion of IFN-γ by anti-GPC3 CAR−iNKT cells (FIG. 4A), CAR iNKT cells in each group co expressing IL-15-IL-15Rα fusion proteins can enhance the secretion capacity of IFN-γ, especially when the intracellular signal domain of CAR iNKT cells is ICD3, the secretion capacity of IFN-γ is about twice that of CAR iNKT with 4-1BB signal domain (FIG. 4B).

### Example 5: Effects of Different Intracellular Signaling Domains and Whether the Fusion Protein IL-15-IL-15Rα is Expressed on the Expression of CAR−iNKT Cells on the Immune Checkpoint LAG-3

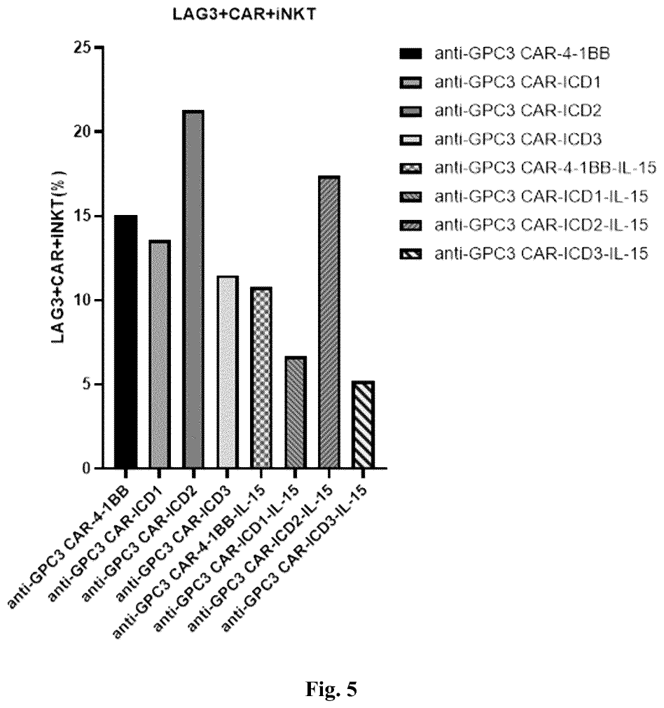

Studies show that in iNKT cells, the increased expression of LAG3 (not PD-1) will damage IFN of iNKT cells-γ The ability to secrete. When CAR iNKT cells in each group were cultured to the 21st day, the CAR iNKT cells in each group (1×10⁵ CAR iNKT cells in each group) and 1×10⁵ Huh-7 cells were taken according to the ratio of effector cells to target cells was 1:1 and co cultured in 0.5 ml X-VIVO (without IL-2 and α-GalCer) for 48 hours, centrifuged at 400×g for 5 minutes, then the supernatant was discarded, the cells were collected and labeled with LAG-3 antibody, and flow cytometry was used to detect the expression of LAG3 on the surface of CAR iNKT in each group. When the intracellular signal domain is ICD1 or ICD3, the proportion of cells expressing LAG-3 will be reduced. When co expressing IL-15-IL-15Rα, the expression ratio of LAG-3 will be reduced (FIG. 5). This shows that ICD1 and ICD3 as intracellular signal domains and co expression of IL-15-IL-15Rα fusion protein can reduce the expression of the co inhibitory molecule LAG3, which may enhance the ability of expression of IFN-γ in CAR iNKT cells, which is consistent with the previous mentioned that ICD1 and ICD3 as intracellular signal domains and co expression of IL-15-IL-15R a fusion protein can increase IFN-γ secretion of CAR iNKT cells (FIG. 4A), thus enhancing the killing ability of corresponding CAR iNKT cells to tumor cells (FIG. 3A).

### REFERENCE

1. https://gco.iarc.fr/today/data/factsheets/populations/900-world-fact-sheets.pdf
2. https://gco.iarc.fr/today/data/factsheets/populations/160-china-fact-sheets.pdf
3. Park, J. W. et al. Global patterns of hepatocellular carcinoma management from diagnosis to death: the bridge study. Liver Int. 35, 2155-2166 (2015).
4. https://www.fda.gov/media/106989/download
5. https://www.fda.gov/media/108458/download
6. Baumhoer D, Tornillo L, Stadlmann S, et al. Glypican 3 expression in human nonneoplastic, preneoplastic, and neoplastic tissues: a tissue microarray analysis of 4,387 tissue samples. Am J Clin Pathol. (2008) 129:899. doi: 10.1309/HCQWPWD50XHD2DW6.
7. Li N, Gao W, Zhang Y F, Ho M. Glypicans as Cancer Therapeutic Targets. Trends Cancer. 2018 November; 4 (11): 741-754. doi: 10.1016/j.trecan.2018.09.004
8. Guo M, Zhang H, Zheng J, et al. Glypican-3: A New Target for Diagnosis and Treatment of Hepatocellular Carcinoma. J Cancer. 2020 Feb. 3; 11 (8): 2008-2021. doi: 10.7150/jca.39972.
9. Crosby, C. M. and M. Kronenberg, Tissue-specific functions of invariant natural killer T cells. Nat Rev Immunol, 2018. 18 (9): p. 559-574.
10. Bennstein, S. B., Unraveling Natural Killer T-Cells Development. Front Immunol, 2017. 8: p. 1950.
11. Georgiev, H., et al., Distinct gene expression patterns correlate with developmental and functional traits of iNKT subsets. Nat Commun, 2016. 7: p. 13116.
12. Lee, Y. J., et al., Lineage-Specific Effector Signatures of Invariant NKT Cells Are Shared amongst gammadelta T, Innate Lymphoid, and Th Cells. J Immunol, 2016. 197 (4): p. 1460-70.
13. Lee, Y. J., et al., Steady-state production of IL-4 modulates immunity in mouse strains and is determined by lineage diversity of iNKT cells. Nat Immunol, 2013. 14 (11): p. 1146-54.
14. Verykokakis, M. and B. L. Kee, Transcriptional and epigenetic regulation of innate-like T lymphocyte development. Curr Opin Immunol, 2018. 51: p. 39-45.
15. Engel, I., et al., Innate-like functions of natural killer T cell subsets result from highly divergent gene programs. Nat Immunol, 2016. 17 (6): p. 728-39
16. Lee, Y. J., et al., Tissue-Specific Distribution of iNKT Cells Impacts Their Cytokine Response. Immunity, 2015. 43 (3): p. 566-78.
17. Gaya, M., et al., Initiation of Antiviral B Cell Immunity Relies on Innate Signals from Spatially Positioned NKT Cells. Cell, 2018. 172 (3): p. 517-533 e20.

1-23. (canceled)

24. A chimeric antigen receptor, comprising a GPC3 antigen binding domain and an intracellular signal stimulation domain.

25. The chimeric antigen receptor according to claim 24, wherein the chimeric antigen receptor further comprises an IL-15-IL-15α fusion protein.

26. The chimeric antigen receptor according to claim 25, wherein the intracellular signal stimulation domain is ICD1, ICD2 or ICD3 with the amino acid sequence SEQ ID Nos. 29, 31 and 33 respectively.

27. The chimeric antigen receptor according to claim 26, wherein the intracellular signal stimulation domain is ICD3 with the amino acid sequence SEQ ID NO.33.

28. The chimeric antigen receptor according to claim 25, wherein the amino acid sequence of the IL-15-IL-15α fusion protein is SEQ ID NO. 7.

29. The chimeric antigen receptor according to claim 25, wherein the GPC3 antigen binding domain is GC33 ScFv containing amino acid sequences SEQ ID NOs. 9 and 11.

30. The chimeric antigen receptor according to claim 29, wherein the GPC3 antigen binding domain is GC33 ScFv with the amino acid sequence SEQ ID NO.13.

31. The chimeric antigen receptor according to claim 24, wherein the chimeric antigen receptor comprises sequentially connected GC33 ScFv, a hinge region, a transmembrane domain, a costimulatory signal domain, ICD1 or ICD2 or ICD3, a CD3 ζ signal conduction domain and a IL-15-IL-15α fusion protein.

32. The chimeric antigen receptor according to claim 31, wherein the amino acid sequences of the hinge region, the transmembrane domain, the costimulatory signal domain, and the CD3 ζ signal conduction domain are SEQ ID NOs. 21, 19, 23 and 35, respectively.

33. An immune cell, wherein the immune cell is transduced into the chimeric antigen receptor according to claim 24, and wherein the immune cell is a T cell, a NK cell or an iNKT cell, and wherein the immune cell is an iNKT cell.

34. A method for treating cancer, comprising administering the immune cells according to claim 33 to a subject in need.

35. The method according to claim 34, wherein the cancer is a cancer with overexpression of GPC3, preferably liver cancer.

36. An expression vector of the chimeric antigen receptor according to claim 24.

37. The expression vector according to claim 36, comprising ICD1, ICD2 or ICD3 nucleic acid sequences with the nucleotide sequences SEQ ID Nos.30, 32 and 34 respectively.

38. The expression vector according to claim 36, comprising IL-15-IL-15α fusion protein nucleic acid sequence with the nucleotide sequence SEQ ID NO.8.

39. An intracellular signal stimulatory molecule, wherein the amino acid sequence is SEQ ID NO. 29, 31 or 33, respectively.

40. A nucleic acid coding sequence of the intracellular signal stimulatory molecule according to claim 39, wherein the nucleic acid coding sequence is SEQ ID No. 30, 32 or 34.

41. A method for manufacture of a chimeric antigen receptor, comprising using the intracellular signal stimulatory molecule according to claim 39.

42. An IL-15-IL-15 α fusion protein, wherein the amino acid sequence is SEQ ID NO. 7.

43. A nucleic acid coding sequence of IL-15-IL-15 fusion protein according to claim 42, wherein the nucleic acid coding sequence is SEQ ID NO. 8.
